(19) United States
(12) Patent Application Publication
(10) Pub. No.: US 2023/0111301 A1
(43) Pub. Date: Apr. 13, 2023

(54) Device for Disinfecting Hands

(71) Applicant: Alexander Roth, Straelen (DE)

(72) Inventor: Alexander Roth, Straelen (DE)

(21) Appl. No.: 17/911,439

(22) PCT Filed: Mar. 1, 2021
(86) PCT No.: PCT/DE2021/000036

§ 371 Date: Sep. 14, 2022

(30) Foreign Application Priority Data
Mar. 25, 2020 (DE) 10 2020 001 927.7

(51) Int. Cl. A61L 2/00; A61L 2/24; A47K 5/12; B60R 15/00

(52) U.S. Cl. CPC A61L 2/0088; A61L 2/24; A47K 5/1217; B60R 15/00; A61L 2202/15; A61L 2202/14

(57) ABSTRACT

The invention relates to a device 1 for disinfecting hands, wherein an outlet for the disinfection liquid 10 is provided in a local external region of a vehicle 20.

In order to provide a vehicle driver of additional occupants the opportunity to disinfect their hands before entering or sitting down in the vehicle, the invention provides that the disinfection liquid 10 exists from a reservoir 2 via at least one outlet opening or spray nozzle 27, which is secured externally on a vehicle 20, when a sensor 12, 23, 28 is triggered. The claimed device makes it possible for a disinfection liquid to exit from a reservoir via an outlet opening or spray nozzle, which is secured externally on a vehicle, when a sensor is triggered, so that the vehicle driver can disinfect their hands from outside. Of course, the same applies to larger vehicles, such as buses or other public means of transport, so that all passengers can disinfect their hands before starting their journey, or, optionally, after their journey has ended.

### CROSS REFERENCE TO RELATED APPLICATIONS

This application is the U.S. national stage of International Application No. PCT/DE2021/000036, filed on Mar. 1, 2021. The international application claims the priority of DE 102020001927.7 filed on Mar. 25, 2020; all applications are incorporated by reference herein in their entirety.

### BACKGROUND

The invention relates to a device for disinfecting hands, wherein an outlet for the disinfection liquid is provided in a local external region of a vehicle.

Disinfectants are needed wherever contamination by bacteria or viruses occurs and further spread is to be prevented. For this purpose, there is a need to provide a disinfectant that can be applied to the palm of a hand, for example. Known disinfection devices are usually found in hospitals, for example, as a wall-mounted device with a large bracket that can be operated with a forearm and allows the disinfectant to emerge from a nozzle via a pump function. Thus, the handle can be pressed down with one arm and the disinfectant can be collected with the second hand to the extent that it can be distributed evenly to both hands. Alternatively, disinfection devices are known which are operated by electric motor, whereby the proximity of a palm is detected by a sensor and a spray jet flows out for a short period. In this case, too, both hands can be disinfected. Such devices have proven themselves especially in hospitals and prevent germs, viruses and bacteria from being transferred from a patient or visitor to the hospital staff, the doctors, but also to the patients. It is not for nothing that the rule applies that especially in such sensitive areas, which of course also include nursing homes, hands should be disinfected before entering and after entering.

In the sign of a pandemic, for example due to the Corona virus, it is essential to be able to carry out a disinfection also in the private area, in order not to infect oneself. This applies in particular after entering publicly accessible rooms, stairwells, use of door handles and other surfaces that are permanently touched by people. It therefore makes sense, for example, to disinfect one's hands before getting into one's car after moving around in public areas. If you have to open the car to reach a spray bottle, the handle or surfaces of the vehicle interior may already be contaminated.

### SUMMARY

The invention relates to a device 1 for disinfecting hands, wherein an outlet for the disinfection liquid 10 is provided in a local external region of a vehicle 20.

In order to provide a vehicle driver of additional occupants the opportunity to disinfect their hands before entering or sitting down in the vehicle, the invention provides that the disinfection liquid 10 exists from a reservoir 2 via at least one outlet opening or spray nozzle 27, which is secured externally on a vehicle 20, when a sensor 12, 23, 28 is triggered. The claimed device makes it possible for a disinfection liquid to exit from a reservoir via an outlet opening or spray nozzle, which is secured externally on a vehicle, when a sensor is triggered, so that the vehicle driver can disinfect their hands from outside. Of course, the same applies to larger vehicles, such as buses or other public means of transport, so that all passengers can disinfect their hands before starting their journey, or, optionally, after their journey has ended.

### DETAILED DESCRIPTION

For the aforementioned reasons, the present invention is based on the task of providing a disinfection option that is specifically intended for a car, in order to enable the disinfection of the hands of the vehicle owner and, if necessary, of third parties, without them having to enter the vehicle.

According to the invention, in order to solve the task, it is provided that the disinfection liquid exits from a reservoir via at least one outlet opening or spray nozzle, which is secured externally on a vehicle, when a sensor is triggered. Further advantageous embodiments of the invention result from the subclaims.

By a device, which is arranged outside of the vehicle, for example a passenger car, a bus, a streetcar or other public means of transport or a truck, the possibility exists for the driver and, if necessary, also for third parties, to disinfect their hands. If several persons use one vehicle, such as police vehicles, fire-brigade or ambulances, e.g., such a device is likewise of advantage. In this case, hand disinfection is of primary importance because the persons concerned can come into contact with viruses in public areas, for example through the use of door handles, elevators, railings or the like, which can lead to contamination due to an infected person who has moved through the premises and touched the surfaces present. In addition, viruses can sometimes remain on such surfaces for a very long time, which means that contamination and thus infection of other persons is also possible over longer periods of time. Insofar as a disinfectant is available, a car driver, for example, can thus disinfect their hands before opening a vehicle and before sitting in the vehicle in order to prevent contamination of the interior. For this purpose, it is provided that the required disinfection liquid is discharged from a reservoir via at least one outlet opening or a spray nozzle, the liquid usually being sprayed under pressure. In this case, the outlet opening or spray nozzle is located on the outside of the vehicle, so that it is possible for the driver of the vehicle to use it. In addition, under special circumstances, use by third parties who also wish to disinfect their hands, may also be possible. This option is appropriate if there is a pandemic and everyone is required to disinfect their hands as quickly as possible, so that a variety of possibilities are available to carry out this disinfection with the aid of a disinfection liquid. This is particularly advantageous for public transport or buses, because many people are confined to in a small space and use, for example, the handgrips or grab rails to hold on to. In the case of larger vehicles, the device can be activated, for example, by the vehicle driver using a selector button, provided that such a device is provided at the factory.

In an embodiment of the invention, it is provided that the device is arranged on at least one exterior rearview mirror, a tailgate or a door of the vehicle. The exterior mirror is ideally suited for this purpose because it protrudes from the vehicle and thus the hands can be held below the exterior mirror in order to carry out the disinfection. The tailgate is also suitable, as it offers the possibility of arranging corresponding outlet openings or spray nozzles, whereby storage containers and other necessary equipment can be installed in the tailgate in an advantageous manner. Alternatively, a door, in particular the door handle of a vehicle, is suitable because the disinfectant solution can be sprayed perpendicular to the door wall with the aid of a spray nozzle.

To enable disinfection on a vehicle, the device has a reservoir for the disinfection liquid, a pump device, at least one sensor and an outlet or spray nozzle. The reservoir holds the disinfectant liquid and the pump device pumps it via corresponding connecting hoses to the spray nozzle or outlet opening. A sensor is used to activate the device so that it is only ever pressurized for a short time in order to maintain the supply of disinfectant liquid for as long a period as possible and not to waste it senselessly.

In a further embodiment of the invention, it is provided that the device can be activated by a vehicle key or a remote control for the vehicle. In this case, the device is initially deactivated and is only activated when the ignition of the vehicle is switched on with the aid of a vehicle key or remote control and the device is thus also switched on at the same time. In this way, it is thus possible to prevent third parties from using the device. Alternatively, it is possible that deactivation is not necessary and that the device is also available to third parties at all times.

In a further embodiment of the invention, it is provided that the device has a motion sensor, a proximity sensor or a temperature sensor which detects a palm of a hand. The sensors serve to control the pump device when the device is activated, in order to let the disinfection liquid flow out of the outlet opening or spray nozzle. The pump device is thus activated only as long as the hands are held in the area of the sensors. For this purpose, the disinfectant liquid is conveyed from the supply container via a supply line to either the exterior mirror, a door lock or the tailgate. Both the reservoir and the pump unit can be installed directly on or in the vehicle, with the sensors for controlling the pump unit being located near the exterior mirror of the door lock or the tailgate. In addition, the sensors have a short range and are not triggered by other activities, for example, when approaching the vehicle normally, but only when the hands are in the immediate vicinity of the sensors.

In a further embodiment, it is envisaged that the device may be factory-fitted in an exterior mirror, in the door handle or in a handle strip of the tailgate. Of course, this can only be provided for new vehicles. Alternatively, it is possible for the device to consist of a kit which is intended for retrofitting to a vehicle. For this purpose, the device is integrated in a housing which can be fixed to an exterior mirror or the tailgate by at least one fixing means. In an advantageous manner, the device can consist of a retrofit kit which can be clamped underneath an exterior mirror, the retrofit kit comprising a reservoir, a pump unit, a voltage source, a sensor and, if necessary, a control unit and thus being ready for use in a self-sufficient manner. Such a retrofit kit with a voltage source is suitable for making the device accessible to everyone, whereby the detection of a palm is carried out with the aid of the sensors and the disinfectant liquid is sprayed under pressure from the outlet openings or spray nozzles with the aid of the pump unit. However, there is also the possibility, since most exterior mirrors are already electrically adjustable and this adjustability only exists when the ignition of the vehicle is switched on, that the voltage supply is provided by the battery of a vehicle and thus the retrofit kit can only start to function when the ignition of the vehicle is switched on.

The focus is on the fact that the device can be activated via the at least one sensor, possibly several sensors, or via a switch, thereby activating the pump unit and enabling disinfection of the hands. Vehicles equipped with such a device can be identified by a sign, sticker or button.

The present invention further aims to offer a method for providing a device which enables disinfection of, for example, the hands in the external area of a vehicle for the vehicle driver or, if necessary, also third parties.

According to the invention, a device is used to solve the method task, which is arranged in the exterior area of a vehicle and is activated via a control unit, whereby the disinfectant solution can be discharged through an outlet opening or spray nozzle in a local exterior area of the vehicle after a control unit has activated the device and detected a palm by a sensor. The disinfectant solution is discharged with the aid of a pump device whenever a sensor has responded. It can be a motion sensor, a proximity sensor or a temperature sensor, which detect a palm. In principle, it is possible to keep the device permanently activated so that the disinfection liquid can be discharged under pressure after a sensor has responded. Alternatively, it is possible to switch on the device via the ignition and thus a disinfection liquid can only emerge after release with a vehicle key or a remote control and the response of a sensor. In this case, the pumping function can be triggered over a preset time frame or for the period of time when at least one sensor responds. As soon as the hands are removed from the area of the sensors, the pump function is deactivated and further leakage of the disinfectant liquid is thus prevented in order to reduce consumption.

The main advantages of the present invention are that a vehicle driver can disinfect their hands outside the vehicle without having to enter the vehicle or sit in the vehicle beforehand. The device for disinfecting the hands is located outside the vehicle and can be activated by a vehicle key or a remote control as far as this is only intended for the vehicle driver. As soon as the hands come near the sensor and the outlet nozzle, the pump unit is activated to apply the disinfection liquid from the spray nozzles to the hands. The period of application can be limited in time or can be dependent on the sensor detection, so that the spraying activity is stopped when the hands are removed from the area of the sensors. Furthermore, it is also possible to offer this disinfection option to third parties. For this purpose, the device is constantly activated and the pump unit is switched on and off by the sensors if hands are detected.

### BRIEF DESCRIPTION OF THE DRAWINGS

The invention is explained again in the following with reference to the figures.

It shows

FIG. 1 shows a block diagram of the device according to the invention for the disinfection of hands and

FIG. 2 two possible arrangements of the device in the area of the exterior rearview mirrors and the tailgate, respectively.

### DETAILED DESCRIPTION OF THE PREFERRED EMBODIMENTS

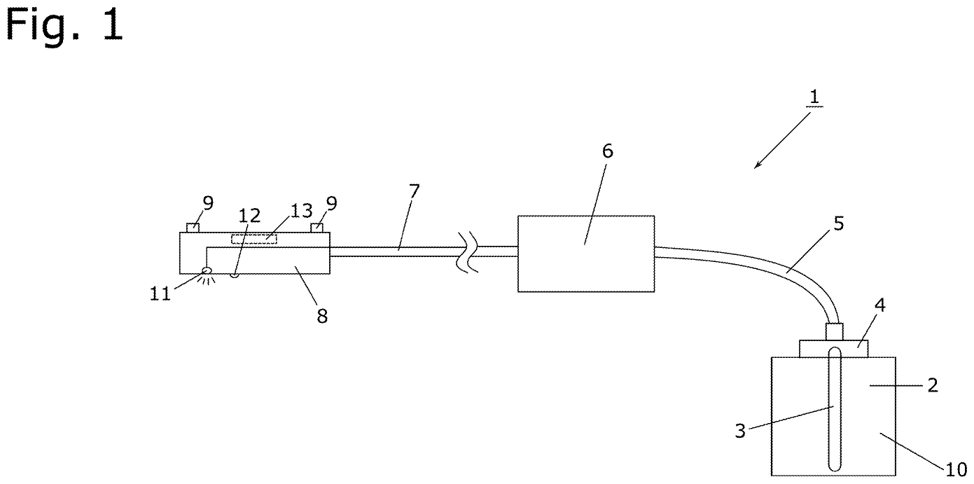

FIG. 1 shows a schematic block diagram of a device 1 for disinfecting hands. The device 1 consists of a reservoir 2 with a suction lance 3 and a screw connection 4 as well as a connecting hose 5, which leads to a pump unit 6 and from the pump unit 6 via a further hose 7 to a housing 8, which can be attached to an exterior mirror, for example, with the aid of two clips 9.

The reservoir 2 contains a disinfection liquid 10 which is conveyed via the hose 5, 7 through the pump unit 6 to the discharge nozzle 11 so that the disinfection liquid 10 can exit. A sensor 12 can be used here to determine whether a hand is located below the discharge nozzle 11, so that the spraying process of the disinfection liquid 10 is activated. The sensor 12 can, for example, consist of a temperature sensor, a motion sensor or an infrared sensor, which enables activation of the pump unit 6 via a control unit 13.

The device 1 is shown schematically and can, for example, be located inside a housing or the individual elements of the device 1 can be accommodated inside the vehicle, with only the discharge nozzles 11 being visible on the outside. Insofar as the discharge nozzles 11 are arranged in the rear area of the vehicle or in the area of the door handles, the further components can thus be accommodated in the tailgate, if necessary also in the door. Insofar as the entire device 1 is provided as a kit, it can be accommodated in a housing which is fastened directly to an exterior mirror with the aid of the clamps 9, the pump unit 6 and the reservoir 2 being integrated within the housing.

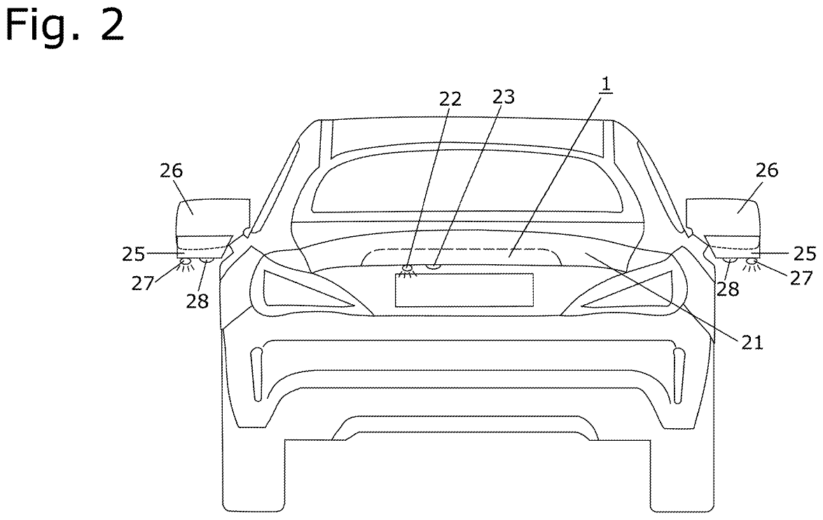

FIG. 2 shows a rear view of a vehicle 20 with possible mounting positions for the device 1 according to the invention.

A first possibility is to provide a device 1 in the tailgate 21 in such a way that a disinfection liquid can exit, for example, in the area of the license plate, whereby a nozzle 22 is provided in the example shown. The nozzle 22 can be located above the license plate, but also in the lateral area of the tailgate 21. In this case, the components of the device 1 can be installed below the tailgate 21, namely a reservoir with disinfectant as well as a pump unit and the necessary connecting hoses to the outlet nozzle 22. A sensor 23 detects the presence of a hand and switches on the pump unit. In this solution, the device 1 is preferably activated via a vehicle key or remote control.

The solution in a tailgate offers the advantage that sufficient storage space is available for the various components. In addition, the device 1 can already be installed at the factory, with a voltage supply coming directly from the battery of the vehicle 20. In this case, it is possible for the device 1 to be activated only when the vehicle 20 is activated with the aid of a key or remote control, i.e. when the ignition is switched on, so that the device 1 is also supplied with voltage and, as soon as a sensor 23 is activated, the disinfection liquid is sprayed with the aid of the pump unit.

An alternative embodiment is that a retrofit kit is installed in a housing 25, wherein the housing 25 can be attached to an exterior mirror 26 using clamps. The housing 25 here accommodates a reservoir, a pump unit and the spray nozzle 27, as well as a sensor 28.

Insofar as a retrofit kit is used, which is accommodated in a housing 25, this can be equipped with an external supply voltage, which supplies a relatively small pump unit with voltage, whereby a sensor 28 is present, which activates the pump unit.

### LIST OF REFERENCE NUMERALS

1 Device

2 Reservoir

3 Suction lance

4 Screw connection

5 Connecting hose

6 Pump unit

7 Hose

8 Housing

9 Clips

10 Disinfection liquid

11 Discharge nozzle

12 Sensor

13 Control unit

20 Vehicle

21 Tailgate

22 Nozzle

23 Sensor

25 Housing

26 Exterior rearview mirror

27 Spray nozzle

28 Sensor

1. Device (1) for disinfecting hands, wherein an outlet for the disinfection liquid (10) is provided in a local external region of a vehicle (20),
characterized in that,
the disinfection liquid (10) exits from a reservoir (2) via at least one outlet opening or spray nozzle (27), which is secured externally on a vehicle (20), when a sensor (12, 23, 28) is triggered.

2. Device (1) according to claim 1,
characterized in that,
the device (1) is secured on at least one exterior rearview mirror (26), one tailgate (21) or one door of the vehicle (20).

3. Device (1) according to claim 1,
characterized in that,
the device (1) consists of a reservoir (2), a pump unit, at least one sensor 12, 23, 28) and an outlet opening or spray nozzle (27).

4. Device (1) according to claim 1,
characterized in that,
the device (1) can be activated by means of a vehicle key or a remote control for the vehicle (20).

5. Device (1) according to claim 1,
characterized in that,
the device (1) contains a movement sensor, a proximity sensor or a temperature sensor, which detects a palm of a hand.

6. Device (1) according to claim 1,
characterized in that,
the reservoir (2) including pump unit (6) is secured on or in the vehicle (20) and transfers the disinfection liquid via a supply to either an exterior rearview mirror (26), a door lock or a luggage compartment door (21).

7. Device (1) according to claim 1,
characterized in that,
the device (1) is secured in a door of the vehicle, an exterior rearview mirror (26) or a tailgate (21).

8. Device (1) according to claim 1,
characterized in that,
the device (1) consists of a construction kit, which is intended to allow the backfitting of a vehicle (20).

9. Device (1) according to claim 1,
characterized in that,
the device (1) is integrated into a housing (8, 25), which is fixable at an exterior rearview mirror (26) or a tailgate (21) with at least one fastening.

10. Device (1) according to claim 1,
characterized in that,
the device (1) consists of a subsequent installation kit, which can be clipped on below an exterior rearview mirror (26), wherein the subsequent installation kit contains a reservoir (2), a pump unit (6), a power supply, a sensor (12, 23, 28) and a control unit (13).

11. Device (1) according to claim 1,
characterized in that,
the device (1) can be activated through the at least one sensor (12, 23, 28) or a switch.

12. Method for the disinfection of hands by means of a device (1), which is provided in an external region of a vehicle (20) and activated through a control unit (13),
characterized in that the disinfection liquid is provided though an outlet opening or spray nozzle (27) which is provided in local external region of a vehicle (20) after a palm of a hand was detected by a sensor (12, 23, 28).

13. Method according to claim 12,
characterized in that,
the control unit (13) is activated by means of a vehicle key or a remote control.

14. Method according to claim 12,
characterized in that,
the control unit (13) contains a sensor (12, 23, 28), which triggers a pump function after the control unit (13) is activated.

15. Method according to claim 12,
characterized in that,
the pump function is triggered for a pre-set time frame or for the period of time during which at least one sensor (12, 23, 28) is activated.
